(19) United States
(12) Patent Application Publication
(10) Pub. No.: US 2018/0315508 A1
(43) Pub. Date: Nov. 1, 2018

(54) METHODS AND APPARATUS FOR DYNAMIC EVENT DRIVEN SIMULATIONS

(71) Applicant: KONINKLIJKE PHILIPS N.V., Eindhoven (NL)

(72) Inventors: Wei WANG, Somerville, MA (US); Yang YANG, Medford, MA (US); Yugang JIA, Winchester, MA (US); Gertjan Laurens SCHUURKAMP, Utrecht (NL); Niels Roman ROTGANS, Eindhoven (NL); Qingxin WU, Malden, MA (US)

(21) Appl. No.: 15/960,894

(22) Filed: Apr. 24, 2018

Related U.S. Application Data
(60) Provisional application No. 62/490,855, filed on Apr. 27, 2017.

(51) Int. Cl. G16H 50/30; G06N 5/02

(52) U.S. Cl. CPC G16H 50/30; G06N 5/022

(57) ABSTRACT

A method and an apparatus for accurately predicting and modeling patient events, such as avoidable admissions, within a healthcare network are disclosed herein. The present disclosure provides systems and methods of predicting and modeling patient events with the use of a constantly updated data set, a sliding windows format, and a random survival forest model. Further, the present disclosure provides methods and systems for accurately predicting and modeling patient events and patient flows amongst various facilities within, and outside of, the healthcare network.

### CROSS-REFERENCE TO PRIOR APPLICATIONS

This application claims the benefit of U.S. Provisional 62/490,855, filed on 27 Apr. 2017. This application is hereby incorporated by reference herein.

### FIELD

The present application relates generally to patient flow simulations. More particularly, the present application relates to systems and methods for generating patient admissions and for validating patient flow simulation on healthcare networks.

### BACKGROUND

Healthcare delivery entities are hospitals, institutions and/or individual practitioners that provide healthcare services to individuals. In recent years, there has been an increased focus on monitoring and improving the delivery of healthcare around the globe. Traditionally, healthcare delivery has been driven by volume, meaning that healthcare delivery entities are motivated to increase or maximize the volume of healthcare services, visits, hospitalizations and tests that they provide.

More recently, there is a growing trend in which healthcare delivery is shifting from being volume driven to being outcome or value driven. This means that healthcare delivery entities are being incentivized to provide high quality healthcare while minimizing costs, rather than simply providing the maximum volume of healthcare. One way in which healthcare delivery entities are being incentivized is by the implementation of payment systems in which healthcare delivery entities (e.g., Accountable Care Organizations (ACOs)) are paid using a pay-for-performance model.

This shift to outcome or value driven service has thus increased the importance of defining, monitoring, and measuring the quality of healthcare, namely focusing on safe, effective, patient-centered, timely, efficient, and equitable healthcare delivery. Healthcare quality measurements are used by emerging outcome or value driven payment models, for example, to benchmark performance against other providers, thereby improving transparency, accountability, and quality; reward or penalize healthcare delivery entities or services that either meet or do not meet certain quality criteria; or conform to medical, environmental, and other like standards or guidelines related to healthcare delivery.

As a result of this shift, healthcare providers have been seeking ways to intuit expected needs of patients and healthcare facilities. This is important for at least two reasons. As a first matter, being able to accurately predict the needs of patients can allow for healthcare networks to maintain facilities with sufficient bandwidth to timely treat patients without long wait times. Secondarily, healthcare provider management has been seeking the capability to predict patient visit patterns in the future. As such there is a need for accurate models that can simulate and predict patient visit patterns that can provide healthcare management the ability to redirect resources, such as staffing and medical supplies. Further, accurate models of patient flows within a network can then, for example, inform strategic operating decisions such as the creation of new facilities and clinic allocations.

While healthcare providers have sought after accurate models, the ability to create, optimize, and validate useful models has been limited at best. For example, often existing models are restricted in terms of the future outlook to a specified period of time, such as three months or six months. Additionally, current models are individual facility focused, rather than network wide. Moreover, existing models often look to patient populations in aggregate, and as a result the model lacks the resolution to accurately predict patient individual visits.

Simulations of patient flows under different scenarios may help the decision makers and stakeholders to gain insight about the system and optimize patient experience. However, simulation of a complex large scale network level can be difficult. Yet, this type of simulation can be instrumental in understanding patient behaviors and optimizing the intricate healthcare system.

Thus, there is a need for improved systems and methods that enable accurate, efficient, and dynamic simulation of future patient visits within healthcare networks.

### SUMMARY

The present disclosure provides methods and systems for accurately predicting and modeling patient events, such as avoidable admissions, within a healthcare network generally. For example, the present disclosure may provide detailed simulations of individual patient cohorts within the network. The present disclosure provides systems and methods of predicting and modeling patient events with the use of a constantly updated data set, a sliding windows simulation, and a random survival forest model.

Various advantages and other features of the structures and methods disclosed herein will become more readily apparent to those having ordinary skill in the art from the following detailed description of certain preferred embodiments taken in conjunction with the drawings which set forth representative embodiments of the present disclosure and wherein like reference numerals identify similar structural elements.

In an exemplary method for predicting avoidable events of patients within a healthcare network, the method includes, receiving by a digital data processor historical claim feed data of avoidable events for a predetermined time period, analyzing with the digital data processor the historical claim feed data with a sliding window model, analyzing with the digital data processor the historical claim feed data with a random survival forest model, predicting the avoidable events using both the sliding window model and the survival forest model; and displaying the predictions made in the predicting step and determining a course of medical treatment for a patient based upon the prediction.

In some embodiments, the historical claim feed data can be a continuous stream of data. Updating the predicting step can be done using new historical claim data. The historical claim feed data can include data pertaining to a single patient cohort type. The historical claim feed data can include whether a patient was admitted to a healthcare facility and the date of admission of the patient to the healthcare facility. The predetermined time period is one of a period of six months, three months, or one month. In some embodiments, the method can further include determining a location within the healthcare network where the patient will likely receive the course of medical treatment.

In one exemplary embodiment, an apparatus for predicting avoidable events of patients within a healthcare network can include, a digital data processor receiving historical claim feed data of avoidable events for a predetermined time period, analyzing with the digital data processor the historical claim feed data with a sliding window model, analyzing with the digital data processor the historical claim feed data with a random survival forest model; predicting with the digital data processor the avoidable events using both the sliding window model and the survival forest model; and a digital display that displays the predictions made the digital data processor and determining a course of medical treatment for a patient based upon the prediction.

In some embodiments, the historical claim feed data can be a continuous stream of data received from a third party. Further, the sliding window model and the random survival forest models can be continuously updated with new historical claim data. Moreover, the historical claim feed data ca includes data pertaining to a single patient cohort type.

In some other embodiments, the historical claim feed data can include whether a patient was admitted to a healthcare facility and the date of admission of the patient to the healthcare facility. Further the predetermined time period can be one of a period of six months, three months, or one month. Moreover, the digital data processor can further determine a location within the healthcare network where the patient will likely receive the course of medical treatment.

It should be appreciated that the present technology can be implemented and utilized in numerous ways, including without limitation as a process, an apparatus, a system, a device, a method for applications now known and later developed or a computer readable medium.

Other aspects and advantages of the invention can become apparent from the following drawings and description, all of which illustrate the principles of the invention, by way of example only.

### BRIEF DESCRIPTION OF THE DRAWINGS

The present application will be more fully understood from the following detailed description taken in conjunction with the accompanying drawings, in which:

FIG. 1 illustrates a flow chart showing a patient decision path for choosing a healthcare facility;

FIG. 2A illustrates a block diagram showing an exemplary simulation system;

FIG. 2B illustrates a block diagram showing a sliding windows simulation system;

FIG. 3 illustrates a random survival forest model;

FIG. 4A illustrates a testing window showing results of the instant prediction model;

FIG. 4B illustrates individual risk monitoring results of the instant prediction model;

FIG. 5 illustrates a block diagram showing an exemplary configuration of a simulation system;

FIG. 6 illustrates exemplary flows of patients between first locations and second locations;

### DESCRIPTION

Certain exemplary embodiments will now be described to provide an overall understanding of the principles of the structure, function, manufacture, and use of the systems and methods disclosed herein. One or more examples of these embodiments are illustrated in the accompanying drawings. Those skilled in the art will understand that the systems and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Further, in the present disclosure, like-numbered components of various embodiments generally have similar features when those components are of a similar nature and/or serve a similar purpose.

The present disclosure provides methods and systems for accurately predicting and modeling patient events, such as avoidable admissions, within a healthcare network generally. For example, the present disclosure may provide detailed simulations of individual patient cohorts within the network. The present disclosure provides systems and methods of predicting and modeling patient events with the use of a constantly updated data set, a sliding windows simulation, and a random survival forest model. Further, the present disclosure provides methods and systems for accurately predicting and modeling patient events and patient flows amongst various facilities within, and outside of, the healthcare network.

The present disclosure provides for simulation models and optimization systems. The simulations and optimizations may generate high quality simulated patient flow, which can be valuable for bettering outcome driven healthcare, strategic analysis, and consulting engagements with big hospital systems. These programs can be implemented individually or collectively as a software suite or a software dashboard. Such a software suite can accept raw data, as discussed below, and output processed information via a console or display that is helpful to healthcare managers, doctors, nurses, and hospital administrators. The present disclosure can leverage large volumes of raw data flows from various sources within a healthcare network to continuously update and tune simulation systems. Various advantages and other features of the structures and methods disclosed herein will become more readily apparent to those having ordinary skill in the art from the following detailed description of certain preferred embodiments taken in conjunction with the drawings which set forth representative embodiments of the present disclosure and wherein like reference numerals identify similar structural elements.

In one embodiment, a system for simulation of patient cohort hospital visits within a healthcare network is disclosed. A patient cohort can be understood as a group of patients all having generally similar medical conditions, such as congestive heart failure, within a single healthcare network. In general, the simulation system includes a database of patient features and historical visits of patients within a cohort between different healthcare facilities; a dynamic survival model; a patient choice model; and a pipe line between the two models. In some embodiments the dynamic survival model can include a sliding windows calculation, as discussed further below. In certain embodiments the patient choice model can model patient behavior based upon hospital reputation, traveling distance, and the waiting time for each hospital. While the systems herein are discussed with reference to patients, and healthcare networks generally, the dataflow and algorithms described herein can be applied to other event-driven networks such as communication network routing systems.

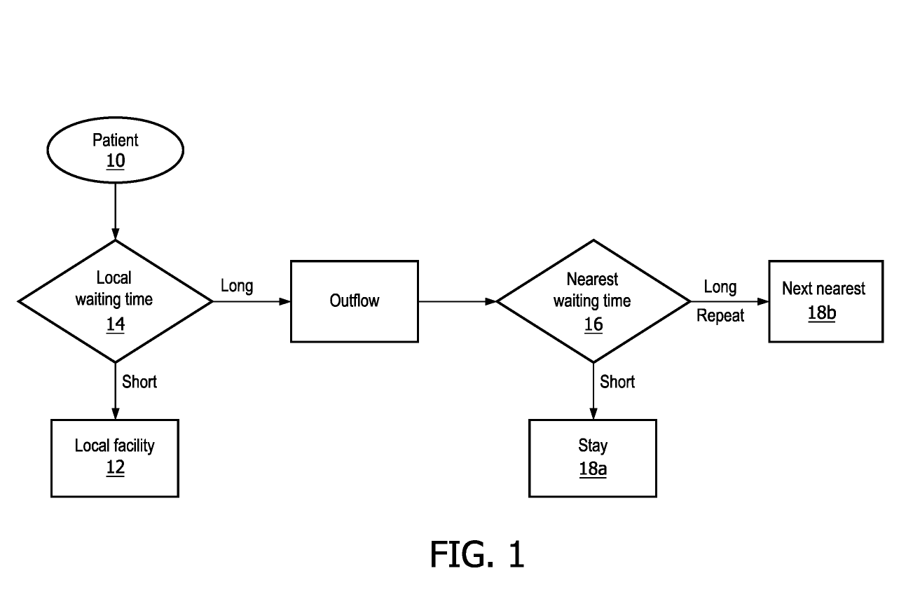

Understanding and modeling patients' behavior can be one important information source for minimizing the number patients leaving a local healthcare facility (“patient outflow”), for optimizing patient experience and life expectancy, and enhancing the overall healthcare network. A decision flow chart is shown in FIG. 1. The flow chart of FIG. 1 can represent the decisions individual patients 10 consider when a visit to a local facility 12 is required. The patient 10 can consider the local wait times 14 at the local facility, if that time is short enough they will likely decide to go to the local facility. However, if the local wait time 14 is too long, the patient will look to the wait time 16 at the next closest facility 18a, this is considered patient outflow. If the wait time at the next nearest facility 18a is acceptable the patient may stay, or alternatively can look to another facility 18b still further away. The patient 10 may balance the length of the wait time with the distance from the facility. Additionally, the patient may consider the reputation of a given facility as yet another factor in the decision making process. Historical behavior models can provide an initial guide for future modeling and simulation of patient flows within the network. With modern Centers for Medicare and Medicaid Services (CMS) and established Accountable Care Organizations (ACOs), there is a wealth of data being collected in digital form for each patient visit. For example, this data can include vast amounts of information pertaining to the networks performance down to individual patient movements through the systems. The historical data can include information regarding the local waiting time at a given medical facility and when patients prefer their local facility. The historical data can indicate, for example, when there is a long wait time a patient will go out of current municipality and go to the nearest one. Alternatively, if the wait time in a given municipality is short, a patient is more likely to stay and receive medical treatment there. Being able to predict this behavior amongst individual patients or patient cohorts can be helpful in balancing network resources and improving patient outcomes.

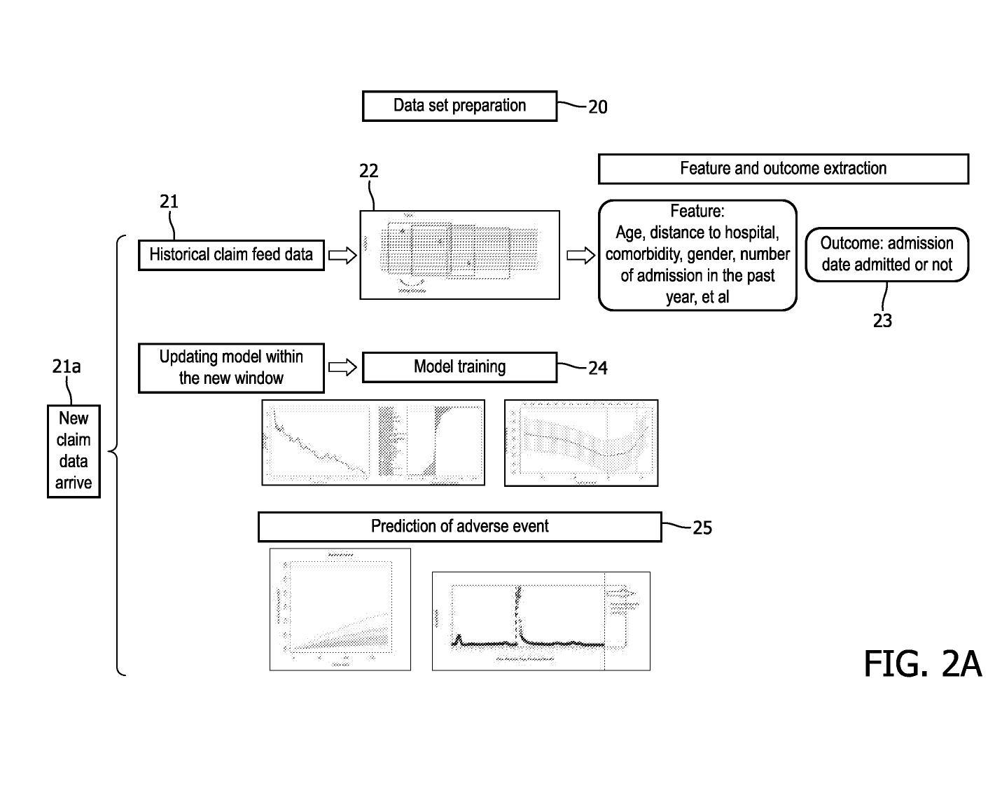

As shown in FIG. 2, as a result of the collaboration between CMS and ACOS, the ACOs may request to receive historical monthly data feed files 21 from their beneficiaries who receive primary care services and have not declined to share their information. Such claim data can contain health condition and visiting episode information for the patient, or beneficiary, which is essential in building a prediction model, that is can be cost-effective to collect and low-latency to obtain. In one example, certain ACOs may serve over 60,000 Medicare beneficiaries. Data for the 60,000 beneficiaries can be collected from the monthly data files called “Accountable Care Organization—Operational System (ACO-OS) Claim and Claim Line Feed (CCLF)”. In one embodiment, the model can look to ACO cohorts of congestive heart failure (CHF) patients from a set time period. Congestive heart failure can, often, be avoided through high quality outpatient care and adherence to care may reduce the rate of occurrence of this event, and thus of hospital admission.

Using an auto-extracted feature set to build the prediction model 20, features can be extracted based on a predefined and extensible entity schema. The candidate feature categories 23 can contain information related to, for example, demographics, chronic conditions, healthcare services, acute exacerbation records, durable medical equipment (DME) utilization, disease-specific procedures and services, medications, location, and cost. These candidate feature categories can be further broken out into individual features, as shown in Table 1. Such features can be extracted at 1 month, 3 months, 6 months, 1 year, and 3 years before the start time point for the survival analysis. The underlying logic is to balance the completeness and the timeliness of the information. As discussed further below, there is an inverse benefit to long term data sets. While a long term data set may often be considered the most “complete,” long term data sets will suffer from the inclusion of stale, or outdated, data. Such stale data may negatively impact any forward looking simulation, though this will depend on the type of data being simulated. For example, the historical total expenditure can be considered as a proxy for both the beneficiary's health condition and the efficiency of the previous healthcare, as such, the prediction model can use a window size of 1 year for more comprehensive information. In contrast, for a given patient's most recent location receiving care, medication history, and acute exacerbation record, more timely information is preferred, and thus a window size of 1, 3 and 6 months, respectively, are used.

TABLE 1

| Feature Category | Features |
|---|---|
| Demographics | Age, Gender, Race. |
| Chronic condition | Any selected chronic conditions; Count of selected |
| | chronic conditions; Charlson Index Score. |
| Healthcare service | Count of a specific healthcare service utilization, |
| | including ED visit, inpatient admission, SNF stay, |
| | HHA stay and outpatient physician visit. |
| Acute exacerbation | Count of ED visit or inpatient admission with |
| record | selected exacerbation conditions. |
| DME utilization | Any DME usage; any oxygen-related DME usage. |
| Disease-specific | Any cardio echo test; any spirometry test; any |
| procedure and | general pulmonary function test. |
| service | |
| Medication | Count of unique prescription. |
| Location | Most recent care location prior to admission, |
| | including home, HHA, SNF, Inpatient and |
| | Outpatient |
| Cost | Total Expenditure |

One goal of the prediction mode is to provide insight to prevent avoidable admission events due to CHF by CMS Ambulatory or primary care sensitive conditions (ACSCs) codes. As noted above, the ability to prevent admission is a goal of modern value driven payment models. The system can record and store, for example, the time to an avoidable admission within half of a year from the beginning of each month from the CCLF data. This raw data 21a can be inputted into the system in real time. If an avoidable admission does not occur within the given period, then it is considered as a right censored in the survival analysis. Censoring data in the survival analysis can be used to insert assumptions into the model. For example, right censoring can be used to assume that a data point is above a certain value, however it is unknown how much above that value it is. The certain value can be assumed to be at least the duration of the window which is being observed. Alternatively, other types of censoring can be used. In the case a patient incurs multiple avoidable admission within a given period, only a few of these event types are likely to happen more than once within a half year period. Thus the prediction mode 25 can consider only the first event of the plurality of events. Doing so can best keep the prediction capability for every beneficiary, even when some of the beneficiaries have more than one additionally balance the model to avoid outliers in the data set from throwing off the prediction capabilities.

The arrival of data 21, 21a in a continuous stream, or a data stream model, can result in a high cost of storage for all of the data. Thus a sliding window technique 22 can be used for continuous data streams because it is deterministic and interpretable. The sliding window technique allows for updated data 21a to be added to the system and older data to be filtered out, for example during a model training mode 24. Thus the older data can be, if needed, deleted to compensate for a growing large volume of data. The sliding window technique, as shown in FIG. 2B, can thus be used to apply statistical algorithms over a finite duration of data—where the data itself is regularly updated. One assumption of imposing sliding window technique, is that the recent data 21a is more informative than old data 21b. As such, the model does not evaluate the entire past history, but rather over sliding windows of the recent data 21a from the streams, as shown in FIG. 2B. Claim data received from ACO may have a potentially large number of participants and features. The volume of claim data may additionally increase monthly. Thus, extraction 23 of the most important and related information from such data in an incremental mode of learning and model adaption can be efficient from a data storage stand point. A sliding window system can have advantages, such as only utilizing a limited amount of memory, short time processing, and single assessment per data window.

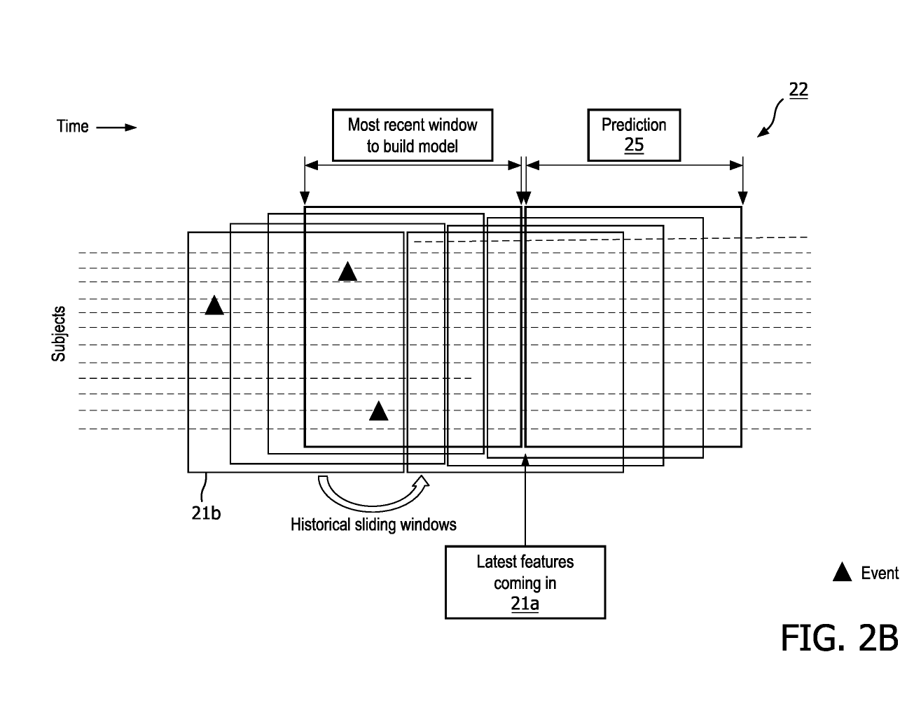

Data can additionally be analyzed by a random survival forest model 30, as shown in FIG. 3. Random survival forests 30 can utilize an ensemble tree to analyze the right censored survival data to predict the expected duration of time until one or more events will occur in the future. For example, the model can predict 35 the time before the next avoidable admission for congestive heart failure patients. The random survival forest is closely related to random forests. As such, random survival forests inherits many of the good properties from random forests. Survival models, in general benefit from using both censored and uncensored data and the time to the event to estimate model parameters, or coefficients. Further, the random survival forest can incorporate bagging of trees 32a, 32b, 32c such that it can improve the learning performance from base learners, or trees. Additionally, with random survival forests, the averaging step 34 of cumulative hazard function H(t) 33a, 33b, 33c can be employed. The final cumulative hazard function H(t) for an individual can come from averaging all the trees in the ensemble. Secondly, the random survival forests are model assumption-free such that it may be more attractive than the parametric and semi-parametric survival models, such as the Cox Proportional model. The random survival forest performs well when there is a highly non-linear or complex relationship between the features and the response. Conversely, the Cox Proportional model does not only require that the relationships are linear, but also requires that the proportional hazard assumption to be held. Like random forest, random survival forests can get its efficiency from the random draw of the bootstrap sample and the random selected predictors. In addition, random survival forests use the log-rank splitting, developed based on a non-parametric log-rank test. For example, R package randomForestSRC can be utilized to fit the random survival forest model. One such R package is available from the Comprehensive R Archive Network (CRAN). See for example, https://cran.r-project.org/web/packages/randomForestSRC/index.html.

In the instant system, the data stream model and random survival forest model can both be leveraged in a single system. A data stream-random survival forests model can allow for the advantages of both data stream model and random survival forest model. Each data stream can correspond to the monthly updating of information from each of the beneficiaries, or patient. It can be assumed that some beneficiaries may withdraw from, or may be added to the plan, thus the model can allow for different number of data streams as a function of time. All the given streams that are present within each month can be referred to as a “window”. Features can be extracted at, and before, the time that the window starts and record the model for the window. Next, the window can be advanced by one month and the process can be repeated until the end of the beneficiaries' time-series. In some examples, the purpose of the models can be to identify the high risk patients for avoidable hospitalization in the following half of a year. Thus, the model can be designed to do an estimation for each window and combine information of the streams from the nearby windows to build a classification model.

The procedure for window combination can be as follows. Assuming the current time window Wt=[t, t+w], for a fixed time point τ, let Wτ denote the set of all times W_{τ}={Wₜ|τ∈[t, t+Δt]}. Different windows can be combined with the use of a hazard function,

$$h ( τ ) = \frac{1}{\# Wt ∈ W τ} {\sum }_{Wt ∈ W τ} h ( τ ) .$$

Thus, the survival function can be expressed as S(t)=exp(−Σh(τ)). Alternatively, a weighting schema can be applied to windows so that the most recent windows are given more weight.

Using the time-varying area under the curve (AUC) and C-statistics to evaluate the model performance at different time points in the validation dataset, the model can then be evaluated. The validation dataset is chosen to be the window of interest since the system is, in general, interested in the prediction power in the window with the most recent feature. Using nearest neighbor estimator to estimate the receiver operating characteristic (ROC) curve can guarantee that sensitivity and specificity are monotone. In addition, the use of cumulative sensitivity and dynamic specificity allows the system to distinguish subjects failing by a given time and those failing after this time. Where, cumulative sensitivity (c,t)=P(Mᵢ(t)>c|Tᵢ≤t); dynamic specificity(c,t)=P(Mᵢ(t)≤c|Tᵢ>t); Mᵢ(t) is the 1-survival function at time t because higher survival function means lower risk, and c is the cutoff point for Mᵢ (t).

Since area under the curve can access the discrimination ability for each time point, it can be a good tool when the system is interested in only a few time points of interest. However, if all time points are of interest, a concordance summary C statistics, a weighted average of AUC, can be used to access the overall model performance. At any time, an unweighted average AUC can additionally be calculated for the scenario when the system is interested in a discrete set of time points of interest.

A large feature pool can be engineered to develop the prediction model for the combined the data stream model and random survival forest model, for example those features outlined in Table 1. As such, a two-step feature selection method can be employed for the models. Certain features with variable importance larger than 0 can be selected into the first round. In a second round of feature selection, the system can perform a nested sequence of models starting with the top variable, followed by a model with the top 2 variables and so on. The feature selection process can be finalized by choosing the features until there is little or no incremental effect of the features. It is possible to have a different feature set for each window.

In use, when the number of windows in the data stream random survival forest (DSRSF) model is one, e.g. when only one batch of data is available, the most recent window can be used to build the random survival forests. The assumption is that the most recent information is the most important and thus the recent window will provide enough prediction power to predict the risk of an event occurring in the next window. In Table 2, the most recent window was used as the validation set. As shown, it can be seen that such a model may perform better in certain windows. For example, the AUC at the 60th day is different from AUC at 180th day. Often the model will provide more stable estimates of AUC at 180th day because there are more events that make up the history before the 180th day than before the 60th day, which can equate to a smaller variance. Using historical windows can additionally help improve the model performance for the earlier period of time within the whole prediction window.

TABLE 2

| Training | | # of | # of | Mean | AUC at | AUC at | | Unweighted |
|---|---|---|---|---|---|---|---|---|
| Window | Validation | subjects in | events in | time to | 60th | 180th | Harrell's | average |
| Start Point | Start Point | validation | validation | event | Days | Days | C statistics | AUC |
| February 2014 | August 2014 | 5175 | 263 | 175.17 | 0.64 | 0.59 | 0.6 | 0.62 |
| March 2014 | September 2014 | 5143 | 250 | 175.14 | 0.62 | 0.66 | 0.6 | 0.62 |
| April 2014 | October 2014 | 5069 | 247 | 175.07 | 0.63 | 0.62 | 0.6 | 0.62 |
| May 2014 | November 2014 | 4988 | 230 | 175.31 | 0.63 | 0.65 | 0.65 | 0.66 |
| June 2014 | December 2014 | 4958 | 225 | 175.3 | 0.67 | 0.68 | 0.65 | 0.69 |
| July 2014 | January 2015 | 4261 | 201 | 175.2 | 0.64 | 0.69 | 0.65 | 0.67 |
| August 2014 | February 2015 | 4233 | 176 | 175.7 | 0.65 | 0.68 | 0.63 | 0.67 |
| September 2014 | March 2015 | 4222 | 172 | 175.85 | 0.64 | 0.67 | 0.65 | 0.65 |

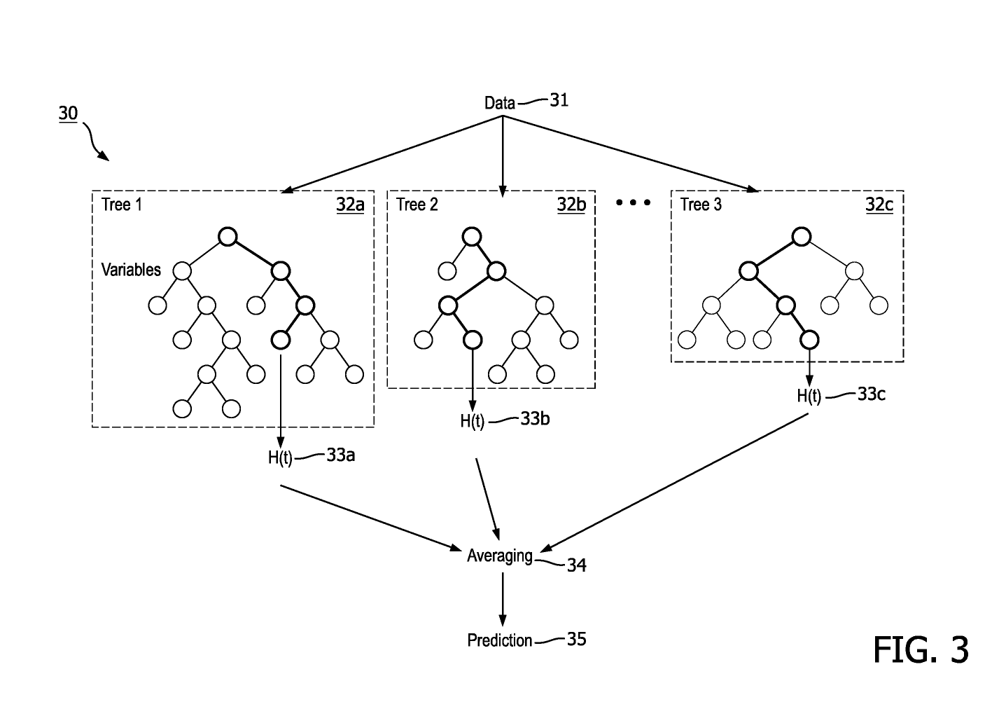

The performance of DSRSF models can be run with different number of windows. The performance of the models can then be compared with a benchmark model. The DSRSF model, in general, is expected to have an overall better performance than the benchmark models. One of the benchmark models can be built with a random survival forest model using all historical outcome data and baseline features. For example, a validation window from March 2015 to August 2015 can be used because it would access most of the historical windows. FIG. 4A illustrates that AUC of DSRSF models 40, 44 are larger than that of benchmark model 42 in general, especially for the first a couple of month. This suggests that the utilization of time-varying feature does help to improve the model performance. In Table 3, it can be seen that by using the DSRSF model, both AUC and C-statistics improve as compared to just using the most recent window or the batch model. For example, by combining three sliding windows, C-statistics can increase from 0.65 to 0.69 and the AUC at 60 days can increase from 0.64 to 0.70. The number of windows can be regarded as a tuning parameter to get the best performance of the DSRSF model. From Table 3 below, it can also be seen that the DSRSF model does not see as drastic of improvements after including more than 3 windows. However, with the inclusion of multiple windows, the DSRSF model can consider the application of data-adaptive weights on a sequence of windows to further improve the prediction performance. For example, windows that are more recent in time can be assigned a larger weight to award more timely information. In one embodiment, for example, the model can assign the weights for the last three windows as, respectively, 0.5, 0.3, and 0.2. Yet another benchmark model to judge the performance of the DSRSF model can be the penalized logistic regression model built upon the most recent window for the 60th day's and 180th day's prediction. As shown in Table 3, the DSRSF model with three windows is at least as good as using a benchmark lasso logistic regression at point predictions. While DSRSF can achieve continuous predictions across time and calculate the risk trajectory of a beneficiary as shown in FIG. 4B, the penalized logistic regression model cannot continuously make predictions due to the nature of the model.

TABLE 3

| | AUC | AUC | Harrell's C | |
|---|---|---|---|---|
| Utilizing the | at 60th | at 180th | statistics for | Unweighted |
| most recent # | Day in | Day in | most recent | average |
| window | Testing | Testing | window | AUC |
| Lasso | 0.66 | 0.71 | NA | NA |
| Logistic(Benchmark) | | | | |
| Batch-mode | 0.59 | 0.67 | 0.59 | 0.61 |
| RSF(Benchmark) | | | | |
| 1 | 0.64 | 0.67 | 0.65 | 0.65 |
| 2 | 0.67 | 0.69 | 0.68 | 0.67 |
| 3 | 0.7 | 0.7 | 0.69 | 0.69 |
| 4 | 0.69 | 0.69 | 0.66 | 0.68 |
| 5 | 0.68 | 0.69 | 0.66 | 0.68 |

As shown in FIG. 4B, the DSRSF model is able to do a risk stratification for each subject based on their survival function at each time point within the window time period. FIG. 4B shows two instant risk curves 46, 48. For example, one risk curve 46 illustrates a randomly selected beneficiary who had an avoidable admission event during the time window and the other risk curve 48 illustrated another beneficiary who did not have such event occur. As illustrated, the subject with overall higher instant risk is the beneficiary who had an avoidable admission 49 within the time window. Based on the historical data, it is shown that the event occurred at the 120th day, which is very close to one of the high peak 46a of the curve. In contrast, when viewing the risk curve 48 for a beneficiary who has not had an adverse event in the given time window, the risk curve is lower. Thus the healthcare network is better able to determine the likelihood of an adverse event occurrence and therefore provide those higher risk patients with better quality healthcare and minimize the associate costs with long term admissions.

The data stream random survival forests (DSRSF) model disclosed herein, seeks to accommodate the growing volume of monthly-updated claim data. The DSRSF model can provide risk classification for every time point in a defined time interval and it does not require fitting multiple models for different time points, such that the comparison at different time points is more consistent within one time window. The DSRSF model is assumption-free due to its non-parametric nature. While it may take a more time to fit a random survival forest model than fitting a parametric survival model, the random survival forest model can select important features, e.g. prescribed medications including depression medications, from a historical data, e.g. data from the last three months, leverage the prediction accuracy through averaging the ensemble tree votes, and behave more unbiased when facing complex relationships between outcome and predictors. When comparing to a random forest model built on all historical data at a batch, the DSRSF model which averages hazard functions from multiple most recent windows can take into account the time-varying and time-sensitive features instead of just using the stale baseline features. As noted above, the window size and number of window can be tuning parameters to get the best performance of the model. By modifying the window sizes and number of windows used in the model the most relevant historical medical information can be utilized for the prediction. In one alternative, it is possible to apply Bayesian framework to the sliding-window platform. Instead of averaging, the model can take into account prior knowledge when analyzing data, turning the data analysis into a process of updating that prior knowledge with biomedical and health-care evidence. The data stream random survival forests model offers a powerful and efficient way to do risk stratification of beneficiaries using data streams in medical area such as monthly updated claim data released from CMS. It can be easily extended to handle a large amount of data and deployed for the practical use. Practical use can include future investments in healthcare facilities and other durable medical equipment across an entire large scale healthcare network.

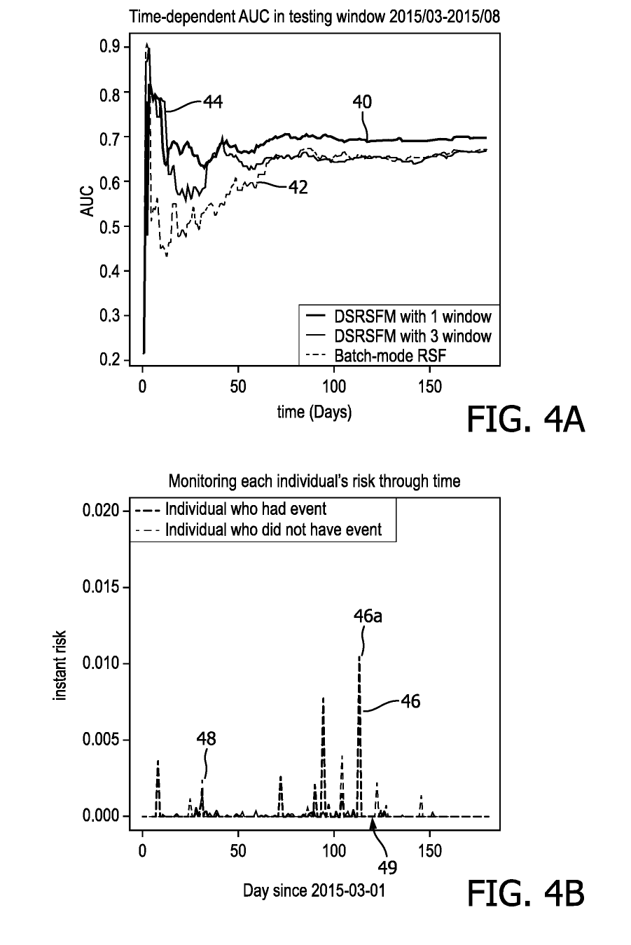

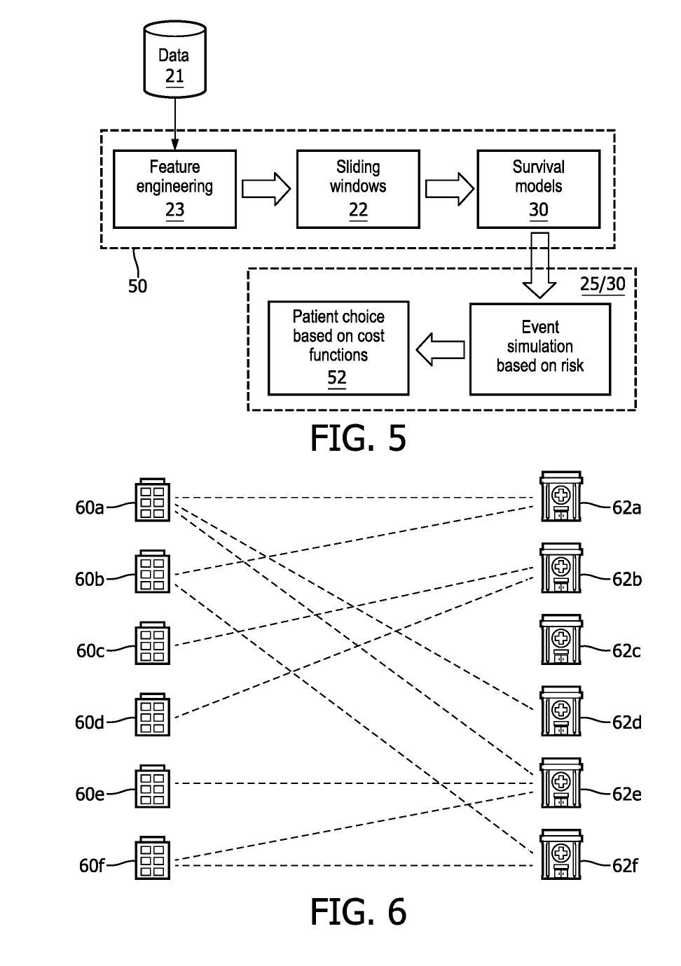

As shown in FIG. 5, data is inputted into the system 50 that can perform feature engineering 23, then formatted as the sliding windows 22, and the survival model 30 to predict the likelihood of an avoidable admission (or other adverse event). Then the adverse event simulation 25, 30 can be implemented based on the risk profile 30. As noted above, once an adverse event occurs, i.e., the need for a hospital visit arises. At such a time the beneficiary, or patient, then makes a choice 52 regarding which hospital to go for the healthcare service. Since the DSRSF model concerns a particular patient cohort, such as congestive heart failure (CHF) patients, it is reasonable to assume that the medical needs amongst the individuals within the cohort are not very different. Therefore, the choice of which hospital 62a-f to attend can be simplified to depend on three factors, hospital reputation, distance to hospital from the patients' locations, residences, or nodes 60a-f, and waiting time at the hospital 62a-f. Mathematically, this can be expressed as a cost function of patient choosing each of the hospital, and the patient would choose the hospital with the lowest cost. Each of the factors, or variables, can be weighted by means of a calculated coefficient to ensure that the model is accurate. The coefficients, or parameters, of the cost function can be fine-tuned by doing a grid-search and then evaluating the resemblance of the simulated data and the historical data, as described in U.S. Patent Application No. 62/490,943, entitled “METHOD AND APPARATUS FOR OPTIMIZATION AND SIMULATION OF PATIENT FLOW”, Docket No. 2016PF01258, filed on an even date herewith, which is incorporated by reference herein in its entirety.

Understanding and modeling patients' future behaviors is important for minimizing patient outflow and for optimizing patients experience and so on. For example, the instant system can aid in the prediction of future preventable readmissions and provide context to doctors who are deciding future medical treatments. Further, the behavior model can directly guide modeling and simulation used for planning further expenditures and expansions. For example, as discussed above, if a patient requires medical attention, they will likely proceed to the local facility in their municipality. Further, if the waiting time at the local facility is short, the patient will prefer the local facility. However, if the wait time to be seen is too long, the patient will look to leave their local municipality and go to a second facility in the next nearest municipality. If the waiting time at the second facility is short, the patient will stay. Alternatively, if the wait time at the second facility is long, the patient will go to a third municipality, and so on. This procedure could be represented by the flowchart illustrated in FIG. 1.

Based on the data format and the patient behavior model, one can use two different methods to solve for optimal allocation of resources, such as MRI machines. The two methods can include a top-down model based optimization procedure and a bottom-up simulation method. Each model can serve as corroboration for the other to validate the output from the system as a whole. Individually, both methods have advantages and disadvantages that serve to balance the other to provide consistent and interpretable results. The recommended allocation can be hard to validate since a “what-if” scenario analysis is often about counterfactual or the future variables. For example, patients' behaviors may change if the healthcare system adds capacity by adding a new facility or by adding capacity at an existing location. Starting from diametric perspectives, simulation- and optimization-based approaches can be used to validate and corroborate each other.

The model based method can, in general, calculate the optimal allocation more easily, however the simulation result is more scale-robust and therefor ultimately more reliable. Thus, business decision makers within the healthcare network are able to make long term planning and allocation decision for the healthcare network. Further, the business decision makers are able to determine if reallocation of resources within the network is needed, thereby optimizing the healthcare network. Therefore, each of the aforementioned goals, minimizing ACO patient leakage, optimizing patients' experience (e.g., waiting time or travel distance), and minimizing overall healthcare expenditures, can be achieved.

Each of the aforementioned systems and models can be applicable in a healthcare network, however, it is contemplated that the modeling and prediction methods disclosed herein can be applicable in a variety of other systems. Moreover, each of the prediction models and algorithms can be part of a software suite or be used individually. The models and algorithms can be processed in the cloud on a remote digital data processor that outputs data, or reports, to end users via a dashboard that is visually depicted as a graphical user interface (GUI). The data or reports can be printed by means of a printer, displayed on a monitor, emailed, or otherwise delivered to end users. The dashboard can be merely an output such that an end user does not have the ability to modify any coefficients, assumptions, or data sets inputted into the system.

While the foregoing description has been directed to specific embodiments, it will be apparent that other variations and modifications may be made to the described embodiments, with the attainment of some or all of their advantages. Accordingly this description is to be taken only by way of example and not to otherwise limit the scope of the embodiments herein. Finally, all publications and references cited herein are expressly incorporated by reference in their entirety.

What is claimed is:

1. A method for predicting avoidable events of patients within a healthcare network, the method comprising:
receiving by a digital data processor historical claim feed data of avoidable events for a predetermined time period;
analyzing with the digital data processor the historical claim feed data with a sliding window model;
analyzing with the digital data processor the historical claim feed data with a random survival forest model;
predicting the avoidable events using both the sliding window model and the survival forest model; and
displaying the predictions made in the predicting step and determining a course of medical treatment for a patient based upon the prediction.

2. The method of claim 1, wherein the historical claim feed data is a continuous stream of data.

3. The method of claim 2 further comprising, updating the predicting step using new historical claim data.

4. The method of claim 1, wherein the historical claim feed data includes data pertaining to a single patient cohort type.

5. The method of claim 4, wherein the historical claim feed data includes whether a patient was admitted to a healthcare facility and the date of admission of the patient to the healthcare facility.

6. The method of claim 1, wherein the predetermined time period is a period of six months.

7. The method of claim 1, wherein the predetermined time period is a period of three months.

8. The method of claim 1, wherein the predetermined time period is a period of one month.

9. The method of claim 1 further comprising,
determining a location within the healthcare network where the patient will likely receive the course of medical treatment.

10. An apparatus for predicting avoidable events of patients within a healthcare network, the apparatus comprising:
a digital data processor receiving historical claim feed data of avoidable events for a predetermined time period, analyzing with the digital data processor the historical claim feed data with a sliding window model, analyzing with the digital data processor the historical claim feed data with a random survival forest model; predicting with the digital data processor the avoidable events using both the sliding window model and the survival forest model; and
a digital display that displays the predictions made the digital data processor and determining a course of medical treatment for a patient based upon the prediction.

11. The apparatus of claim 10, wherein the historical claim feed data is a continuous stream of data received from a third party.

12. The apparatus of claim 11 wherein the sliding window model and the random survival forest models are continuously updated with new historical claim data.

13. The apparatus of claim 10, wherein the historical claim feed data includes data pertaining to a single patient cohort type.

14. The apparatus of claim 13, wherein the historical claim feed data includes whether a patient was admitted to a healthcare facility and the date of admission of the patient to the healthcare facility.

15. The apparatus of claim 10, wherein the predetermined time period is a period of six months.

16. The apparatus of claim 10, wherein the predetermined time period is a period of three months.

17. The apparatus of claim 10, wherein the predetermined time period is a period of one month.

18. The apparatus of claim 10 wherein the digital data processor further determines a location within the healthcare network where the patient will likely receive the course of medical treatment.
